(19) United States
(12) Patent Application Publication
(10) Pub. No.: US 2008/0207923 A1
(43) Pub. Date: Aug. 28, 2008

(54) Pure DNT-maleate and methods of preparation thereof

(76) Inventors: Santiago Ini, Haifa (IL); Mili Abramov, Givataim (IL)

Correspondence Address: KENYON & KENYON LLP, ONE BROADWAY, NEW YORK, NY, 10004, US

(21) Appl. No.: 11/981,318

(22) Filed: Oct. 30, 2007

Related U.S. Application Data
(63) Continuation-in-part of application No. 11/525,336, filed on Sep. 21, 2006.
Application No. 11/525,336 is a continuation-in-part of application No. 11/809,730, filed on May 31, 2007.
(60) Provisional application No. 60/719,880, filed on Sep. 22, 2005.
(60) Provisional application No. 60/761,583, filed on Jan. 23, 2006.
(60) Provisional application No. 60/771,069, filed on Feb. 6, 2006.
(60) Provisional application No. 60/809,977, filed on May 31, 2006.

(51) Int. Cl. C07D 333/20

(52) U.S. Cl. 549/75

(57) ABSTRACT

(S)—N,N-Dimethyl-3-(1-naphthalenyloxy)-3-(2-thienyl)propanamine maleate (DNT-maleate) and polymorphs of DNT-maleate, compositions of DNT-maleate and its polymorphs, processes for the preparation of DNT-maleate and its polymorphs, and processes for the preparation of duloxetine hydrochloride from DNT-maleate are provided. Processes for preparing chemically pure duloxetine and chemically pure duloxetine intermediates are also provided. In addition, chemically pure DNT and salts thereof are provided.

### CROSS-REFERENCE TO RELATED APPLICATIONS

This application is a continuation-in-part of U.S. application Ser. No. 11/525,336, filed Sep. 21, 2006, which claims the benefit of priority to U.S. provisional application Ser. Nos. 60/719,880 filed Sep. 22, 2005; 60/761,583 filed Jan. 23, 2006; and 60/771,069 filed Feb. 6, 2006, hereby incorporated by reference. This application is also a continuation-in-part of U.S. application Ser. No. 11/809,730, filed May 31, 2007, which claims the benefit of priority to U.S. provisional application Ser. No. 60/809,977, filed May 31, 2006, hereby incorporated by reference.

### FIELD OF THE INVENTION

The invention is directed to (S)—N,N-Dimethyl-3-(1-naphthalenyloxy)-3-(2-thienyl)propanamine maleate (“DNT-maleate”), an intermediate in the synthesis of duloxetine. In particular, the invention is directed to enantiomerically and chemically pure DNT-maleate. The invention is also directed to the solid state chemistry of DNT-maleate, as well as processes for preparing DNT-maleate and for converting DNT-maleate into duloxetine HCl. In addition, the invention is directed to chemically pure duloxetine.

### BACKGROUND OF THE INVENTION

Duloxetine hydrochloride (“duloxetine HCl”) is a dual reuptake inhibitor of the neurotransmitters serotonin and norepinephrine. It is used for the treatment of stress urinary incontinence (SUI), depression, and pain management. It is commercially available in the U.S. under the tradename CYMBALTA®. Duloxetine hydrochloride is known by the chemical name (S)-(+)-N-methyl-3-(1-naphthalenyloxy)-3-(2-thienyl) propanamine hydrochloric acid salt, and has the following structure.

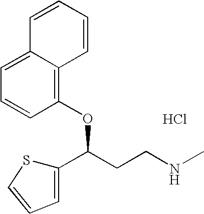

Duloxetine, as well as processes for its preparation, is disclosed in U.S. Pat. No. 5,023,269. European patent No. 457559 and U.S. Pat. Nos. 5,491,243 and 6,541,668 also provide synthetic routes for the preparation of duloxetine. U.S. Pat. No. 5,023,269 discloses preparing duloxetine by reacting (S)-(−)-N,N-Dimethyl-3-(2-thienyl)-3-hydroxypropanamine with fluoronaphthalene (Stage a), followed by demethylation with phenyl chloroformate or trichloroethyl chloroformate (Stage b) and basic hydrolysis (Stage c), according to the following scheme.

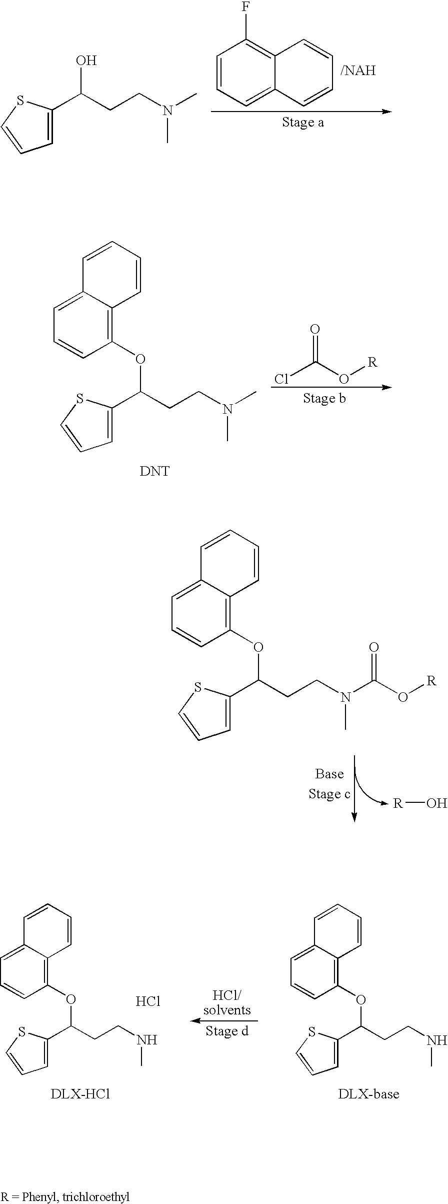

The conversion of duloxetine to its hydrochloride salt in ethyl acetate (Stage d) is described in U.S. Pat. No. 5,491,243 and in Wheeler, W. J., et al, J. Label. Cpds. Radiopharm, 1995, 36, 312.

As illustrated in the above scheme, DNT is an intermediate in the preparation of duloxetine. DNT has an N,N-dimethyl group instead of a secondary amine.

U.S. Pat. No. 5,023,269 describes the preparation of DNT-oxalate from DNT. See U.S. Pat. No. 5,023,269, Example 1. The oxalate salt of U.S. Pat. No. 5,023,269 is problematic for use on an industrial scale because it is prepared from oxalic acid, which is highly toxic. Therefore, there is a need in the art to prepare duloxetine HCl in relatively high purity with a process that is suitable for use on an industrial scale.

Stereochemical purity is of importance in the field of pharmaceuticals, where many of the most prescribed drugs exhibit chirality, and the two isomers exhibit different potency. Furthermore, optical purity is important since certain isomers may actually be deleterious rather than simply inert. Therefore, there is a need to obtain the desired enantiomer of duloxetine HCl in high enantiomeric purity.

A composition of DNT is often contaminated with an enantiomeric impurity (enantiomer R). This enantiomeric impurity generally carries over to the final pharmaceutical product, i.e., duloxetine HCl. The Applicants have found that formation of the oxalate salt as carried out in European patent No. 457559 does not reduce the amount of the enantiomeric impurity. Thus, there is a need in the art for a process that reduces the quantity of enantiomer R present in DNT.

Chemical purity is also of importance in the field of pharmaceuticals. Like any synthetic compound, duloxetine can contain extraneous compounds or impurities that can come from many sources. They can be unreacted starting materials, by-products of the reaction, products of side reactions, or degradation products. Impurities in duloxetine or any active pharmaceutical ingredient (API) are undesirable, and, in extreme cases, might even be harmful to a patient being treated with a dosage form of the API in which a sufficient amount of impurities is present. Furthermore, the undesired enantiomeric impurities reduce the level of the API available in the pharmaceutical composition.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, it must be analyzed for purity, typically, by HPLC or TLC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. In the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

(+)—N-methyl-3-(1-naphthalenyloxy)-3-(3-thienyl)propanamine is disclosed by Olsen B. A et al, as an impurity obtained in the preparation of duloxetine (J. Lib. Chrom. & Rel. Technol, 1996, 19, 1993). U.S. Pat. No. 4,956,388 discloses the synthesis of N,N-dimethyl-3-(1-naphthalenyloxy)-3-(3-thienyl)propanamine (“DNT-ISO3”) and N-methyl-3-(1-naphthalenyloxy)-3-(3-thienyl)propanamine (DLX-ISO3”).

Thus, there is also a need in the art for processes for preparing duloxetine which are suitable for use on an industrial scale and result in a product with high chemical purity.

### SUMMARY OF THE INVENTION

In one embodiment, the invention provides a compound (DNT-maleate) having the following formula:

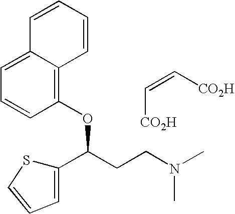

In another embodiment the invention provides a process for preparing a pharmaceutically acceptable salt of duloxetine, comprising combining DNT, a solvent selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof with maleic acid to form a reaction mixture, precipitating DNT-maleate from the reaction mixture, optionally recrystallizing the DNT maleate from ethyl acetate, converting the DNT maleate to DNT, converting the DNT to duloxetine, and converting the duloxetine to the pharmaceutically acceptable salt of duloxetine.

In another embodiment, the invention provides a crystalline form of DNT-maleate:

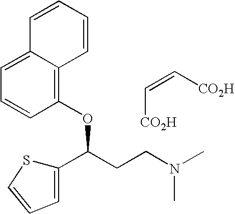

characterized by a powder XRD pattern with peaks at about 9.4°, 14.0°, 18.7°, 23.3°, and 24.9° 2θ±0.2°2θ.

In another embodiment, the invention provides a crystalline form of DNT-maleate:

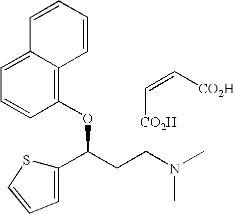

characterized by a powder XRD pattern with peaks at about 14.4°, 18.5°, 23.1°, 27.2°, and 31.3°2θ±0.2°2θ.

In another embodiment, the invention provides a crystalline form of DNT maleate:

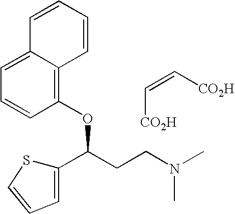

characterized by a powder XRD pattern with peaks at about 9.4°, 18.7°, 23.4°, and 25.3°2θ±0.2°2θ.

In another embodiment, the invention provides a process for preparing a pharmaceutically acceptable salt of duloxetine, comprising combining DNT, a solvent selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof with maleic acid to form a reaction mixture, precipitating DNT-maleate from the reaction mixture, recrystallizing the DNT maleate from ethyl acetate, converting the DNT maleate to DNT, converting the DNT to duloxetine, and converting the duloxetine to the pharmaceutically acceptable salt of duloxetine.

In another embodiment, the invention provides a process for preparing a pharmaceutically acceptable salt of duloxetine, comprising combining DNT, a solvent selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof with maleic acid to form a reaction mixture, precipitating DNT-maleate from the reaction mixture, recrystallizing the DNT maleate from ethyl acetate, converting the DNT maleate to DNT, converting the DNT to duloxetine, and converting the duloxetine to the pharmaceutically acceptable salt of duloxetine.

In one embodiment, the present invention provides a process for preparing duloxetine (or a salt thereof) or a pharmaceutical composition thereof having less than about 2% by HPLC of N-methyl-3-(1-naphthalenyloxy)-3-(3-thienyl) propanamine (DLX-ISO3) comprising measuring the level of 3-acetyl thiophene in a batch of 2-acetyl thiophene, selecting a batch having less than about 2% of 3-acetyl thiophene; and synthesizing duloxetine (or a salt thereof) or a pharmaceutical composition thereof from the batch.

In another embodiment, the present invention provides a process for preparing (+)-N,N-dimethyl-3-(1-naphthalenyloxy)-3-(2-thienyl)propanamine (DNT) having less than about 1% by HPLC of (+)-N,N-dimethyl-3-(1-naphthalenyloxy)-3-(3-thienyl)propanamine (DNT-ISO3) comprising measuring the level of 3-acetyl thiophene in a batch of 2-acetyl thiophene, selecting a batch having less than about 2% of 3-acetyl thiophene; and preparing DNT or a salt thereof from the batch.

In another embodiment, the present invention provides a process for preparing duloxetine (or a salt thereof) or a pharmaceutical composition thereof having less than about 1% by HPLC of N-methyl-3-(1-naphthalenyloxy)-3-(3-thienyl) propanamine (DLX-ISO3) comprising measuring level of DNT-ISO3 or a salt thereof in a batch of (+)-N,N-dimethyl-3-(1-naphthalenyloxy)-3-(2-thienyl)propanamine (DNT) or salt thereof, selecting a batch having less than about 1% of DNT-ISO3 or a salt thereof; and synthesizing duloxetine (or a salt) or a pharmaceutical composition thereof from the batch.

In another embodiment, the present invention provides a process for preparing (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC comprising: (a) combining (S)—N,N-dimethyl-3-(1-naphthalenyloxy)-3-(2-thienyl)propanamine and maleic acid to form a reaction mixture; (b) precipitating (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC from the reaction mixture; and (c) recovering the (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC from the reaction mixture.

In another embodiment, the present invention provides (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC prepared by the above-described process.

In another embodiment, the present invention provides a process for preparing (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC comprising: (a) preparing (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC by the above-described process; and (b) converting the (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC into (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC; and, optionally, (c) converting the (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC into a salt of (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC.

In another embodiment, the present invention provides (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC prepared by the above-described process.

In another embodiment, the present invention provides (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC. Preferably, the (S)—N,N-dimethyl-3-(1-naphthalenyloxy)-3-(2-thienyl)propanamine or a salt thereof contains less than about 0.5%, more preferably less than 0.14%, even more preferably less than about 0.07%, even more preferably less than about 0.04%, and most preferably essentially 0.0% of N,N-dimethyl-3-(1-naphthalenyloxy)-3-(3-thienyl)propanamine by HPLC.

### BRIEF DESCRIPTION OF THE FIGURES

FIG. 1 illustrates the powder X-ray diffraction pattern for DNT-maleate Form Ma1;

FIG. 2 illustrates the powder X-ray diffraction pattern for DNT-maleate Form Ma2; and

FIG. 3 illustrates the powder X-ray diffraction pattern for DNT-maleate Form Ma3.

### DETAILED DESCRIPTION OF THE INVENTION

The invention meets a need in the art by providing a process for preparing N,N-dimethyl-3-(1-naphthalenyloxy)-3-(2-thienyl)propanamine (“DNT”) or a salt thereof, which is an intermediate in the synthesis of duloxetine, substantially free of the impurity N,N-dimethyl-3-(1-naphthalenyloxy)-3-(3-thienyl)propanamine (“DNT-ISO3”). Preferably the DNT salt is a maleate, succinate, fumarate, benzensulfonate or di-P-toluoyl-L-tartrate salt, and most preferably a maleate salt.

(S)—N,N-dimethyl-3-(1-naphthalenyloxy)-3-(2-thienyl)propanamine maleate (“DNT-maleate”) can be represented by the formula C₂₃H₂₅NO₅S and the following structure:

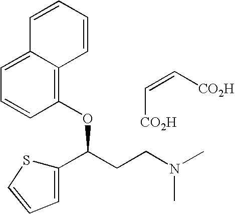

DNT-maleate is preferably isolated as a solid, and, more preferably as a crystal. The use of DNT-maleate as an intermediate salt for preparation of DNT is advantageous because it allows one to obtain such hydrochloride salt in relatively high purity without many of the drawbacks of the oxalate salt.

Use of the DNT-maleate salt provides an enantiomeric cleaning effect not observed with the oxalate salt. The cleaning effect results from the process of obtaining DNT-maleate which produces a greater ratio of the S enantiomer relative to the R enantiomer, than was present in the DNT starting material.

DNT-maleate can be characterized by data selected from: ¹H NMR (400 MHz, CDCl₃ d6) δ(ppm): 8.32 (d, J=8.3 Hz, 1H), 7.82 (d, J=8.0 Hz, 1H), 7.55 (m, 2H), 7.46 (d, J=8.2 Hz, 1H), 7.31 (m, 2H), 7.14 (d, J=3.4 Hz, 1H), 6.98 (t, J=4.3 Hz, 1H), 6.87 (d, J=7.7 Hz, 1H), 6.31 (s, 2H), 5.85 (t, J=6.1 Hz, 1H), 3.16 (m, 2H), 2.83 (s, 6H), 2.69 (m, 1H), 2.57 (m, 1H); 13C {1H}NMR (100 MHz): δ 171.0, 153.8, 144.1, 137.0, 136.0, 129.1, 128.5, 128.0, 127.2, 127.1, 127.0, 126.9, 123.1, 122.8, 108.6, 74.8, 55.9, 44.35, 34.9; and FAB MS: m/z 312 ([M-H]+, 100%).

A general scheme for the synthesis of DNT or a salt thereof is as follows:

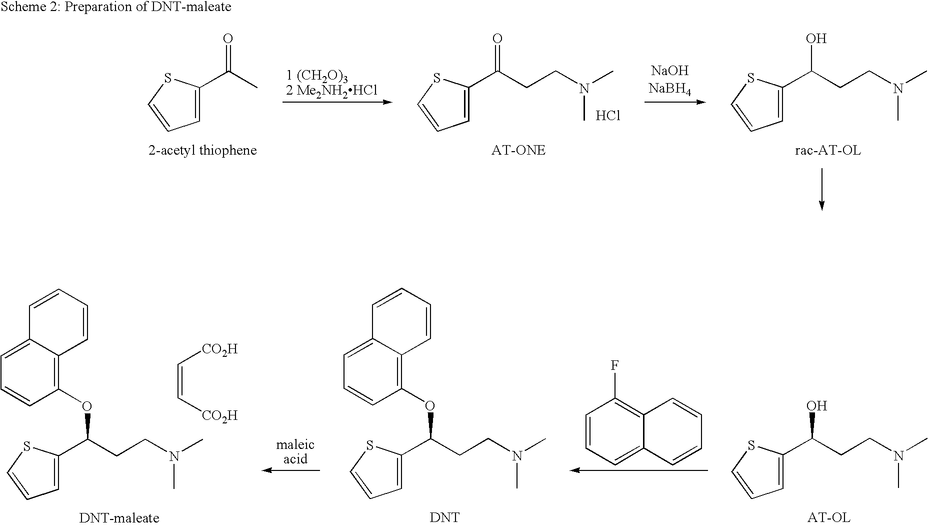

More specifically, the synthesis can comprise: (1) combining 2-acetylthiophene, paraformaldehyde, dimethylamine and a solvent to obtain a mixture containing 3-dimethylamino-1-(2-thienyl)-1-propanone (“AT-ONE”); (2) combining the mixture with a strong base, reducing agent and a C₁-C₈ alcohol or a mixture of C₁-C₈ alcohol with water to obtain a racemic mixture of N,N-dimethyl-3-(2-thienyl)-3-hydroxypropanamine (“AT-OL”); (3) combining the racemic mixture of AT-OL with mandelic acid in a solvent selected from the group consisting of: water, C₁₋₈ alcohols, C₃₋₈ ketones, C₂₋₈ alkyl esters, C₅₋₈ aromatic hydrocarbons, and mixtures thereof to obtain enantiomerically pure AT-OL; (4) combining the enantiomerically pure AT-OL with halonaphthalene and a base to obtain DNT; and, optionally, (5) converting the obtained DNT to a DNT salt, such as the maleate salt.

The dimethylamine used can be introduced into the reaction mixture either in its based form, or as a salt. Preferably, the dimethylamine is dimethylamine HCl.

The solvent used in step (a) may be any inert solvent. Typically, polar organic solvent can be used. Preferably, C₁-C₈ alcohol are used, most preferably, the solvent is isopropyl alcohol (IPA).

Preferably, the combination of 2-acetylthiophene, paraformaldehyde source, dimethylamine and the solvent is heated to obtain the mixture containing AT-ONE. More preferably, the combination is heated to reflux.

Typically, the mixture containing AT-ONE is filtered, to obtain a solid, and further combined with a strong base, sodium borohydride and a polar aprotic solvent.

Preferably, the strong base is selected from the group consisting of alkali metal hydroxide and alkali metal alkoxides. More preferably, the strong base is potassium hydroxide (KOH), sodium methoxide, or sodium hydroxide (NaOH).

The strong base may be added portionwise in order to increase the chemical yield.

Typically, the strong base is combined with a solution of AT-ONE in the solvent. Preferably, the solution is cooled prior to the addition of the base.

In one specific embodiment, a solution of AT-ONE in methanol and water is cooled to a temperature of about 0° C. and further combined with sodium hydroxide.

Preferably, the reducing agent is selected from the group consisting of: sodium borohydride (NaBH₄), lithium borohydride (LiBH₄), lithium aluminum hydride (LiAlH) and selectride. More preferably, the reducing agent is NaBH₄.

The mixture containing AT-OL obtained, after combining with the reducing agent, is a racemic mixture, which is further subjected to chiral resolution.

Preferably, the organic solvent used for the chiral resolution is selected from the group consisting of isopropanol, methyl iso-butyl ketone, and toluene.

Combining of the racemic mixture of AT-OL, mandelic acid and the solvent can be carried out at a temperature of about room temperature to about reflux temperature. Preferably, racemic AT-OL is combined with mandelic acid in the solvent at a temperature of about 50° C.

The reaction mixture may be further heated to accelerate the chiral resolution process. Preferably, the heated reaction mixture is maintained after a precipitate appears, more preferably for about 45 minutes.

Preferably, the heated reaction mixture is cooled to a temperature of about 15° C. to about 25° C., to obtain a precipitate.

The obtained enantiomerically pure AT-OL can be either (S)-AT-OL or (R)-AT-OL, depending on the enantiomerically pure acid introduced into the reaction. For example, when (S)-mandelic acid is used, (S)-AT-OL is obtained.

The halonaphthalene is preferably 1-fluoronaphthalene or 1-chloronaphthalene.

In one specific embodiment, DNT is prepared by providing a solution of a base selected from the group consisting of: alkali metal hydroxide, sodium and alkali metal alkoxides, AT-OL and polar aprotic solvent at a temperature of from about 15° C. to about the reflux temperature of the solvent; combining the solution with 1-fluoronaphthalene or 1-chloronaphthalene, with or without a phase transfer catalyst, to obtain a mixture; heating the mixture to a temperature of from about room temperature to about the reflux temperature of the solvent and recovering DNT.

The DNT may be converted to a salt of DNT by a process comprising combining DNT and the respective acid to obtain the desired salt. Preferred salts are: maleate, succinate, fumarate, benzensulfonate and Di-P-toluoyl-L-tartrate. Most preferably, the salt is a maleate salt, and the acid is maleic acid.

The present invention also provides a process for preparing DNT maleate. DNT maleate may be prepared by combining DNT and maleic acid to create a reaction mixture. DNT maleate forms in such reaction mixture through contact of DNT with maleic acid.

In one embodiment, a solution or a suspension of DNT in a solvent is combined with maleic acid to form a reaction mixture, followed by recovery of the DNT-maleate salt from the reaction mixture. The maleic acid may be either added as a solid or as a solution or suspension in an organic solvent. The organic solvent present in the reaction mixture is preferably selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof. Preferably, the solvent is acetone, n-butanol (“n-BuOH”), ethyl acetate, methyl t-butyl ether (“MTBE”), toluene or water. More preferably, the solvent is ethyl acetate, acetone, or n-BuOH.

In one embodiment, DNT, maleic acid and at least one solvent are combined to form a reaction mixture at about room temperature. The amount of maleic acid present in such reaction mixture is preferably to the point of saturation. DNT maleate then precipitates out of such mixture. Such precipitation may occur on its own or be induced. The reaction mixture may be stirred before, during or after precipitation.

In another embodiment, the reaction mixture of maleic acid, DNT, and solvent is heated. Heating may be carried out from about room temperature to about the reflux temperature of the solvent. Preferably, the reaction mixture is heated to about the reflux temperature of the solvent. Preferably, the reaction mixture is maintained, while heating, for about 15 minutes. DNT maleate forms in the reaction mixture. The reaction mixture may then be cooled to facilitate precipitation of the DNT-maleate. The reaction mixture is generally cooled to a temperature of about 50° C. or less, preferably about room temperature, and more preferably about 15° C., to facilitate precipitation. The reaction mixture may be stirred before, during or after precipitation.

The above embodiments, with or without heating, may be carried out without a solvent. In this method, DNT is used both as a reagent and a solvent; Maleic acid and DNT are combined to form a reaction mixture followed by precipitation of the DNT-maleate.

The resulting precipitate from any of the above embodiments may be recovered by conventional techniques, such as filtration. The precipitate may be dried under ambient or reduced pressure, or elevated temperature. In one embodiment, the precipitate is dried at room temperature at a pressure of less than about 100 mm Hg.

The DNT-maleate can also be prepared in different polymorphic forms. Polymorphism, the occurrence of different crystal forms, is a property of some molecules and molecular complexes. A single molecule, such as DNT-maleate may give rise to a variety of crystalline forms having distinct crystal structures and physical properties like melting point, X-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula, yet having distinct physical properties that can be advantageous in certain applications compared to other crystalline forms of the same compound or complex. Therefore, processes for the preparation of polymorphic forms of DNT-maleate are desirable.

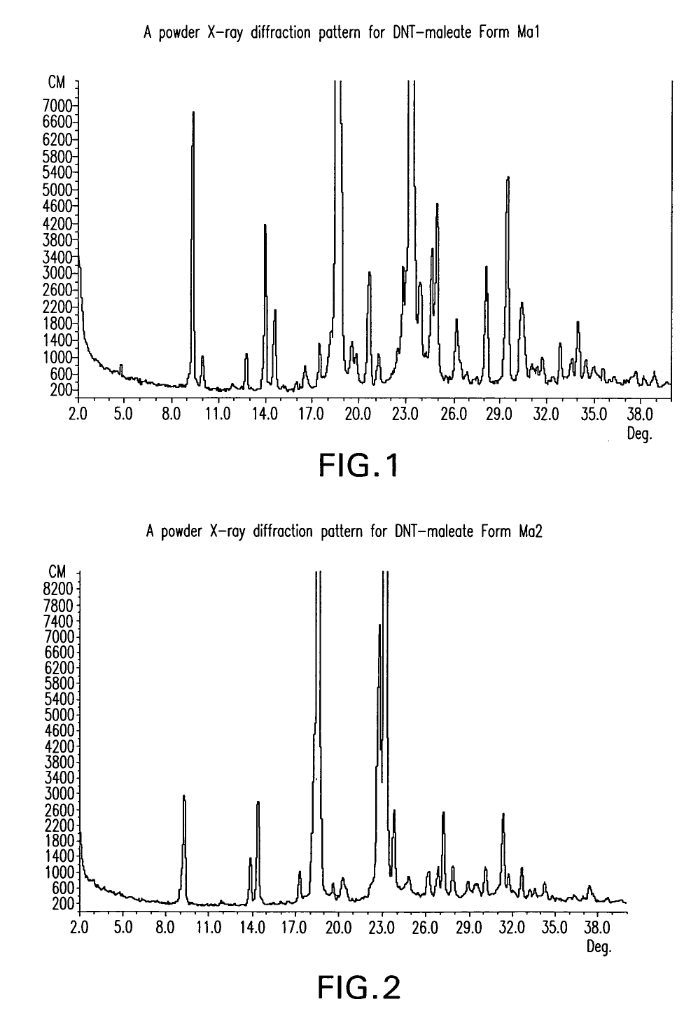

One such crystalline form of DNT-maleate, herein defined as Form Ma1, is characterized by a powder XRD pattern with peaks at about 9.4°, 14.0°, 18.7°, 23.3°, and 24.90° 2θ±0.2° 2θ. The crystalline Form Ma1 may be further characterized by X-ray powder diffraction peaks at about 20.6°, 24.6°, and 29.4° 2θ±0.2°±2θ. DNT-maleate Form Ma1 can also be characterized by an X-ray powder diffraction pattern substantially as depicted in FIG. 1.

Form Ma1 may be prepared by precipitation from a C₃-C₇ ketone, preferably acetone. This process is preferably carried out at about room temperature. In this embodiment, DNT, maleic acid and the ketone are combined to form a mixture, followed by recovery of the DNT maleate. Generally, maleic acid is added to a solution of DNT in the ketone. The maleic acid may also be added as a solution in the ketone to a solution of the DNT in the ketone. The DNT maleate precipitates from the reaction mixture. The reaction mixture may be stirred before, during or after precipitation. The precipitate may be recovered by conventional techniques, such as filtration. The precipitate may be dried under ambient or reduced pressure, or elevated temperature. In one embodiment, the precipitate is dried at room temperature at a pressure of less than about 100 mmHg.

Another crystalline form of DNT-maleate, herein defined as Form Ma2, is characterized by a powder XRD pattern with peaks at about 14.4°, 18.5°, 23.1°, 27.2°, and 31.3° 2θ±0.2°2θ. The crystalline Form Ma2 may be further characterized by X-ray powder diffraction peaks at about 13.9°, 20.3°, and 26.3°2θ±0.2°2θ. DNT-maleate Form Ma2 can also be characterized by an X-ray powder diffraction pattern substantially as depicted in FIG. 2.

Form Ma2 may be prepared by precipitation from a wide range of solvents including C₃-C₇ ester, C₁-C₈ alcohol, C₃-C₈ ether, water or mixtures thereof, preferably n-butyl alcohol, ethyl acetate, MTBE, water and mixtures thereof.

In one embodiment, DNT, maleic acid and at least one solvent listed above are combined to form a reaction mixture at about room temperature. The amount of maleic acid present in such reaction mixture is preferably to the point of saturation. DNT maleate Form Ma2 then precipitates out of such mixture. Such precipitation may occur on its own or be induced. The reaction mixture may be stirred before, during or after precipitation.

In another embodiment, maleic acid and DNT in a solvent are heated to form a reaction mixture. The amount of maleic acid present in such reaction mixture is preferably to the point of saturation. Heating may be carried out from about room temperature to about the reflux temperature of the solvent. DNT maleate forms in the reaction mixture. The reaction mixture may then be cooled to facilitate precipitation of the DNT maleate Form Ma2. Cooling is generally carried to a temperature of about 50° C. or less, preferably about room temperature, to facilitate precipitation. The reaction mixture may be stirred before, during or after precipitation.

The resulting Form Ma2 precipitate may be recovered by conventional techniques, such as filtration. The precipitate may be dried under ambient or reduced pressure, or elevated temperature. In one embodiment, the precipitate is dried at room temperature at a pressure of less than about 100 mmHg.

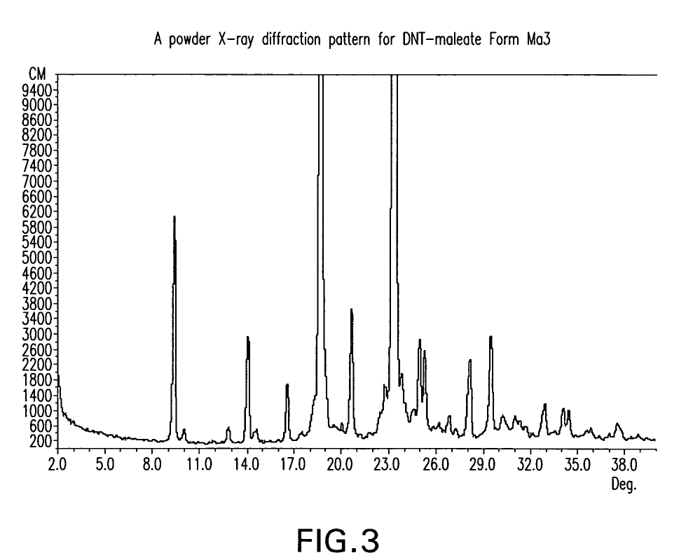

A further crystalline form of DNT-maleate, herein defined as Form Ma3, is characterized by a powder XRD pattern with peaks at about 9.4°, 18.7°, 23.4°, and 25.3°2θ±0.2°2θ. The crystalline Form Ma3 may be further characterized by X-ray powder diffraction peaks at about 14.0°, 20.6°, 24.9°, 28.1°, and 29.5°2θ±0.2°2θ. DNT-maleate Form Ma3 can also be characterized by an X-ray powder diffraction pattern substantially as depicted in FIG. 3.

Form Ma3 may be prepared by precipitation from a heated C₃-C₇ ketone reaction mixture, preferably acetone. In this embodiment, DNT, maleic acid and the ketone are combined to form a mixture. Generally, maleic acid and DNT in the ketone are heated, followed by cooling to facilitate precipitation. The temperature for heating is generally about room temperature to about reflux temperature of the solvent. The reaction mixture may be stirred before, during or after precipitation. Cooling is generally carried to a temperature of less than about 50° C., preferably less than about room temperature, most preferably, at 4° C. The resulting precipitate may be recovered by conventional techniques, such as filtration. The precipitate may be dried under ambient or reduced pressure, or elevated temperature. In one embodiment, the precipitate are dried at room temperature at a pressure of less than about 100 mmHg.

Preferably, the DNT-maleate resulting from any of the above processes is present in a composition (such as a batch) having a polymorphic purity of at least about 10 percent by weight, more preferably, at least about 25 percent by weight, and most preferably at least about 50 percent by weight of a single crystalline form.

Converting DNT into DNT maleate by any of the above-described processes can also lower the amount of the undesired R-enantiomer present in the DNT. Such reduction in the level of undesired R-enantiomer can be calculated according to the following formula:

$$( 1 - \frac{\% {R}_{DNT \text{-} Maleate}}{\% {R}_{DNT}} ) \times 100$$

Preferably the molar amount of the R-enantiomer of DNT-maleate, compared to the DNT starting material, is less than about 50 percent, more preferably less than about 20 percent and even more preferably less than about 4 percent of the molar amount present in such DNT starting material.

The enantiomeric purity can be even further increased by repeating the above process, i.e., converting the DNT-maleate into DNT and then converting the DNT back into DNT-maleate. Preferably, after repetition of the process, the DNT-maleate contains an undetectable amount of the undesired R-enantiomer. In other words, the process may further comprise combining DNT-maleate with a base, combining the DNT-base with maleic acid to form a reaction mixture, precipitating DNT-maleate from the reaction mixture, and recovering the DNT-maleate.

To decrease the level of the R-enantiomer of DNT-maleate even further, the DNT-maleate prepared by the processes of the present invention may be crystallized from one or more polar solvents, such as C₁₋₈ alcohols, e.g., n-butanol, C₃₋₇ esters, e.g., ethyl acetate, water, and mixtures thereof. Preferably, the DNT-maleate of the invention is crystallized from ethyl acetate. The crystallization may be performed by dissolving DNT-maleate in the organic solvent, preferably at a temperature of about room temperature to about reflux temperature, followed by cooling. The obtained DNT-maleate is recovered by any method known in the art, such as filtering, and may be further washed and dried.

In one embodiment of the present invention, successive crystallization from ethyl acetate is carried out until the enantiomeric impurity is no longer detectable. A solvent as listed above may be used instead of ethyl acetate. The DNT-maleate of the present invention preferably contains less than about 50%, more preferably less than about 15%, even more preferably less than about 5%, and even more preferably less than about 0.04% of enantiomeric impurity. Most preferably such impurity is undetectable by HPLC.

The DNT-maleate of the invention, including Form Ma1, Form Ma2 and Form Ma3, will generally have a maximal particle size of less than about 500 μm, preferably less than about 300 μm, more preferably less than about 200 μm, and most preferably less than about 100 μm. A particularly preferred crystalline Form Ma3 of DNT-maleate has a maximal particle size of less than about 50 μm. The particle size of DNT-maleate crystalline forms may be measured by methods including, but not limited to sieves, sedimentation, electrozone sensing (coulter counter), microscopy, and Low Angle Laser Light Scattering (LALLS).

Preferably, the DNT or a salt thereof prepared by any of the above processes is substantially free of N,N-dimethyl-3-(1-naphthalenyloxy)-3-(3-thienyl)propanamine (“DNT-ISO3”) or its salt. As used herein, and with reference to DNT or a salt thereof, substantially free means containing less than about 1% DNT-ISO3, as measured by HPLC, preferably less than about 0.5%, even more preferably about 0.14%, even more preferably less than about 0.07% and even more preferably, less than about 0.04%, and most preferably below the detection limit; i.e., the DNT or salt thereof contains essentially 0.0 percent DNT-ISO3 within the error limits of the detection of HPLC. Preferably, the DNT is (S)-DNT.

The DNT-maleate of the present invention is useful as an intermediate in the preparation of pharmaceutically salts of duloxetine, particularly the hydrochloride salt. The conversion can be carried out by combining DNT-maleate, water, a base such as ammonium hydroxide, and toluene to obtain a two phase system, separating the organic phase containing DNT and toluene, and converting the DNT to duloxetine HCl. The DNT-maleate used in this process is preferably the DNT-maleate prepared as described above. As such, it has a low content of the R-enantiomer and is preferably substantially free of DNT-ISO3, and, therefore, the duloxetine HCl obtained from the DNT-maleate of the invention also has a decreased R-enantiomer content and is preferably substantially free of DNT-ISO3.

The conversion of DNT to a pharmaceutically acceptable salt of duloxetine may be performed by any method known in the art, such as the one described in U.S. Pat. No. 5,023,269 or in co-pending U.S. patent application Ser. No. 11/318,365, filed on Dec. 23, 2005 (published as U.S. publication No. 2006/0194869), for making duloxetine HCl. Preferably, the conversion is performed by dissolving DNT in an organic solvent, and combining it with an alkyl haloformate. That step will yield duloxetine alkyl carbamate, which can be combined with an organic solvent and a base, to yield duloxetine. The duloxetine may then be converted to a pharmaceutically acceptable salt. More preferably, the conversion is performed by dissolving DNT in a water immiscible organic solvent; adding alkyl chloroformate at a temperature of about 5° C. to less than about 80° C. to obtain duloxetine alkyl carbamate, combining the duloxetine alkyl carbamate with an organic solvent and a base; maintaining the reaction mixture at reflux temperatures for at least 1 to 3 hours; cooling, and adding water and an additional amount of an organic solvent; recovering duloxetine; combining the duloxetine with a solvent; adding hydrochloric acid until a pH of about 3 to about 4 is obtained; maintaining the reaction mixture to obtain a solid residue; and recovering duloxetine HCl.

Pharmaceutical compositions can be made using the pharmaceutically acceptable salts of duloxetine from the processes described above. A pharmaceutical composition may comprise a pharmaceutically acceptable salts of duloxetine from the processes described above, and a pharmaceutically acceptable excipient. Preferably, a pharmaceutical composition can be made by combining the duloxetine HCl produced by the above method with a pharmaceutically acceptable excipient. These pharmaceutical compositions contain less than about 50%, more preferably less than about 15%, even more preferably less than about 5%, and even more preferably less than about 0.04% of enantiomeric impurity. Most preferably such impurity is undetectable by HPLC.

In addition to the active ingredient(s), the pharmaceutical compositions of the present invention contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be died using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate, or sodium acetate.

Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, suppositories, sachets, troches, and losenges, as well as liquid syrups, suspensions, and elixirs.

The dosage form of the present invention may be a capsule containing the composition, preferably a powdered or granulated solid composition of the invention, within either a hard or soft shell. The shell may be made from gelatin, and, optionally, contain a plasticizer such as glycerin and sorbitol, and an opacifying agent or colorant.

The active ingredient and excipients may be formulated into compositions and dosage forms according to methods known in the art.

A composition for tableting or capsule filling can be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted or other excipients may be added prior to tableting, such as a glidant and/or a lubricant.

A tableting composition can be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step In addition, the present invention provides a process for preparing duloxetine substantially free of the impurity (+)—N-methyl-3-(1-naphthalenyloxy)-3-(3-thienyl) propanamine, referred to herein as DLX-ISO3, and represented by the formula:

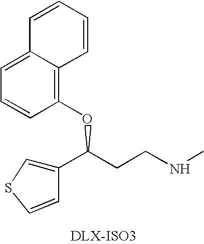

Applicants have found that batches of the starting material in the synthesis of duloxetine, specifically those of 2-acetylthiophene, are contaminated with the impurity 3-acetylthiophene. Further, at each step in the synthesis of duloxetine, this impurity is also transformed. By detecting and controlling amount of this impurity in the beginning of the synthetic process, we have found that it is possible to eliminate or reduce the corresponding 3-thienyl impurities from being present in upstream intermediates and products.

Preferably the batches of 2-acetylthiophene contain less than about 2%, more preferable less than about 1% and most preferably less than about 0.5% by HPLC of 3-acetylthiophene. In one embodiment, a batch having about 0.56% of the impurity is chosen.

Use of these batches for synthesis results in duloxetine and its pharmaceutical compositions, particularly tablets, being substantially free of DLX-ISO3. As used herein, and with reference to duloxetine, substantially free means containing less than about 2% DLX-ISO3, as measured by HPLC. Preferably duloxetine contains less than about 0.5%, more preferably less than about 0.14%, even more preferably less than about 0.07% and even more preferably, less than about 0.04%, and most preferably below the detection limit; i.e., the duloxetine contains essentially 0.0 percent DLX-ISO3 within the error limits of the detection of HPLC.

Use of these batches for synthesis also results in DNT or its salt being substantially free of DNT-ISO3 or its salt.

After selecting a desirable batch of 2-acetyl thiophene, duloxetine is synthesized. The synthesis generally comprises reacting 2-acetylthiophene with paraformaldehyde and dimethylamine, or a salt thereof, reduction with a reducing agent, such as sodium borohydride, chiral resolution with mandelic acid, reaction with a halonaphthalene and reaction with maleic acid, as described above.

In another embodiment, a batch of DNT is selected. Preferably the batch contains less than about 0.5% of DNT-ISO3 or salt thereof, more preferably less than about 0.14% of DNT-ISO3 or salt thereof and most preferably about 0.0% of DNT-ISO3 or salt thereof.

The DNT salt obtained, such as the maleate, can be converted to duloxetine by subjecting the DNT salt to basic hydrolysis. This process can comprise demethylation of the DNT with alkyl chloroformate, followed by basic hydrolysis.

In one embodiment the conversion of DNT to duloxetine is performed as described in U.S. Pat. No. 5,023,269 or in U.S. publication No. 2006/0194869. Preferably, the conversion is performed by a process comprising: dissolving DNT in an organic solvent to obtain a solution; combining the solution with an alkyl haloformate to obtain duloxetine alkyl carbamate; and combining the duloxetine alkyl carbamate with an organic solvent and a base to obtain duloxetine. More preferably, the conversion is performed by a process comprising dissolving DNT in a water immiscible organic solvent to obtain a first solution; adding alkyl chloroformate to the first solution at a temperature of about 5° C. to less than about 80° C. to obtain duloxetine alkyl carbamate; combining the duloxetine alkyl carbamate with an organic solvent and a base to obtain a mixture; heating the mixture to reflux temperature and maintaining the mixture at reflux temperature for at least 1 to 3 hours; cooling the mixture and adding water and an additional amount of an organic solvent to the mixture to obtain duloxetine. Processes for preparation of duloxetine are also disclosed in U.S. publication No. 2006/0194869 and U.S. publication No. 2006/0270731, incorporated herein by reference.

If a commercially available batch does not meet the purity requirements for selection, it may be possible to improve the purity level before use in the synthetic process. For example, if the measured 2-acetylthiophene batch contains more than about 2% of 3-acetylthiophene, it may be purified according to, e.g., the process described in U.S. Pat. No. 5,371,240, incorporated herein by reference.

Additionally, if the measured DNT batch contains more than about 1% of the DNT-ISO3 impurity, it may be purified by converting it to a salt of DNT, and basifying the obtained salt to obtain DNT, substantially as described in examples 6 and 7 below for the maleate salt.

Similarly, if the measured DNT-salt batch contains more than about 1% of the DNT-ISO3 salt impurity, it may be purified by basifying to obtain DNT, followed by converting the obtained DNT to the DNT salt. Most preferably, the salt is a maleate salt.

These steps may be repeated in order to decrease the impurities content even more.

The following non-limiting examples are merely illustrative of the preferred embodiments of the present invention, and are not to be construed as limiting the invention, the scope of which is defined by the appended claims.

### EXAMPLES

### Instruments

X-Ray powder diffraction (XRD) data was obtained using a Scintag X-ray powder diffractometer model X'TRA equipped with a Cu-tube solid state detector. A round standard aluminum sample holder with rough zero background quartz plate with a cavity of 25 (diameter)×0.5 mm (depth) was used. The scanning parameters included: range: 2° to 40° 2θ; scan mode: continuous scan; step size: 0.05°; and a rate of 5°/minute.

### HPLC Method for Measuring Enantiomeric Purity:

| Column: | Diacel Chiral OD 250 × 4.65 μm |
|---|---|
| Eluent: | Hexane (900 ml):IPA (100 ml):DBA (2 ml) |
| Flow: | 1 ml/minute |
| Detection: | 230 nm |
| Sample cone: | 0.5 mg/ml |
| Sample vol: | 100 μl |
| Column temp: | 20° C. |
| Detection limit: | 0.02% |

### HPLC Method for Measuring Chemical Purity:

| Column: | Hypersyl Gold (150 × 4.6 5μ) |
|---|---|
| Mobile phase: | (A) 63% ((NH₄)H₂PO₄ (0.02 M) pH-2.5):37% |
| | (78% MeOH:22% THF) |
| | (B) 20% ((NH₄)H₂PO₄ (0.02 M) pH-2.5):80% ACN |
| Gradient: | From 0 to 15 min (A) isocraticaly |
| | From 15 to 60 min (B) increases from 0 to 75% |
| Detection: | 230 nm |
| Flow: | 1 ml/min |
| Detection limit: | 0.02% |

### Preparation of DNT Maleate

### Example 1

A solution of 7.45 g maleic acid in 50 ml acetone was added to a solution of 20 g DNT in 25 ml of acetone at 25° C., and stirred at the same temperature for one hour. The resulting solid was filtered off, washed with 10 ml of acetone, and dried in a vacuum oven (10 mm Hg) at room temperature for 48 hours, resulting in 18.65 g of DNT maleate (chemical yield: 68%). The product was analyzed by XRD and found to be Form Ma1.

### Examples 2-5

Maleic acid (1.5 g) was added to a solution of 4 g of DNT (2.30% enantiomer R) dissolved in 40 ml of an appropriate solvent, and stirred for about 1 hour. The resulting solid was filtrated, and washed with 8 ml of the appropriate solvent. The product was analyzed by XRD and HPLC, and the results are set forth in

TABLE 1

XRD

| Example | Solvent | % yield | % R | dry¹ | wet |
|---|---|---|---|---|---|
| 2 | n-BuOH | 48.5 | 0.96 | Ma2 | Ma2 |
| 3 | ethyl acetate | 80 | 0.40 | Ma2 | Ma2 |
| 4 | MTBE | 91 | 2.20 | Ma2 | Ma2 |
| 5 | water | 75 | 0.99 | Ma2 | Ma2 |

¹Results obtained after drying in a vacuum oven (10 mm Hg) at 50° C. for 16 hours.

### Examples 6-8

Maleic acid (1.5 g) was added to a solution of 4 g of DNT (2.30% enantiomer R) dissolved in 40 ml of an appropriate solvent, and the mixture was heated to reflux for about 10 minutes. After cooling to room temperature (except where is indicated), the mixture was stirred for an additional 1 hour, filtrated, and washed with 8 ml of the appropriate solvent. The results are described in Table 2:

TABLE 2

| | | | | XRD | |
|---|---|---|---|---|---|
| Example | Solvent | % yield | % R | dry¹ | wet |
| 6 | n-BuOH | 76 | 0.24 | Ma2 | Ma2 > Ma1 |
| 7 | ethyl acetate | 86 | 0.19 | Ma2 | Ma2 |
| 8² | acetone | 50 | 0.30 | Ma3 | Ma3 |

¹Results obtained after drying in a vacuum oven (10 mm Hg) at 50° C. for 16 hours.
²Cooling to 4° C.

### Example 9

Maleic acid (2 g) was added to a solution of 4 g of DNT (2.30% enantiomer R) dissolved in 40 ml of MTBE, and the mixture was heated to reflux for about 10 minutes. After cooling to room temperature, the mixture was stirred for an additional 1 hour, filtrated, and washed with 8 ml of MTBE. After drying in a vacuum oven (10 mm Hg) at 50° C. for 16 hours, 4.8 g (88% yield) of product were obtained. The product was analyzed by XRD and found to be Form Ma2 before, and, after drying, the level of the R-enantiomer found in the product was 2.26.

### Example 10

Maleic acid (1.1 g) was added to a solution of 3 g of DNT (2.30% enantiomer R) dissolved in 40 ml of water, and the mixture was heated to reflux for about 10 minutes. After cooling to room temperature, the mixture was stirred for an additional 1 hour, filtrated, and washed with 8 ml of water. After drying in a vacuum oven (10 mm Hg) at 50° C. for 16 hours, 2.9 g (70% yield) of product were obtained, containing 0.19% of enantiomer R. The product was analyzed by XRD, and found to be Form Ma2 before and after the drying.

### Example 11

Maleic acid (1.2 g) was added to a solution of 4 g of DNT (16% enantiomer R) dissolved in 40 ml of ethyl acetate heated to reflux. The mixture was maintained at reflux for about 10 minutes, cooled to room temperature, and stirred for an additional 1 hour. The resulting solid was filtrated, and washed with ethyl acetate. After drying in a vacuum oven (10 mm Hg) at 50° C. for 16 hours, 3.4 g (62% yield) of DNT-maleate were obtained containing 7.35% of enantiomer R.

### Example 12

Maleic acid (0.2 g) was added to a solution of 0.67 g of DNT (7.35% enantiomer R) dissolved in 7 ml of ethyl acetate heated to reflux. The mixture was maintained at reflux for about 10 minutes, cooled to room temperature, and stirred for an additional 1 hour. The resulting solid was filtrated, and washed with ethyl acetate. After drying in a vacuum oven (10 mm Hg) at 50° C. for 16 hours, 0.74 g (80% yield) of DNT-maleate were obtained containing 1.04% of enantiomer R.

### Example 13

### Crystallization of DNT-Maleate

Ten ml of ethyl acetate were heated to reflux, and slowly added to a mixture of 1 g of DNT-maleate (7.35% enantiomer R) in 10 ml of ethyl acetate until dissolution. The solution was cooled to room temperature, and stirred for an additional one and half hours. The resulting solid was filtrated, and washed with ethyl acetate. After drying in a vacuum oven (10 mm Hg) at 50° C. for 16 hours, 0.69 g (69% yield) of DNT-maleate were obtained containing 0.3% of enantiomer R.

### Example 14

### Crystallization of DNT-Maleate

A mixture of 0.5 g of DNT-maleate (0.3% enantiomer R) in 10 ml of ethyl acetate was heated to reflux, and stirred for 3 hours. The solution was cooled to room temperature, and stirred for an additional one and half hours. The resulting solid was filtrated, and washed with ethyl acetate. After drying in a vacuum oven (10 mm Hg) at 50° C. for 16 hours, 0.47 g (94% yield) of DNT-maleate were obtained, having no detectable amounts of enantiomer R.

### Example 15

### Preparation of DNT

A 22 percent solution of ammonium hydroxide (1 ml) was added to a mixture of 1.5 g of DNT-maleate in 15 ml toluene and 15 ml water. The mixture is stirred at 25° C. for 20 to 30 minutes, the organic phase was separated and washed with water (3×30 ml), and the solution was evaporated to dryness to give 0.67 g of DNT.

### Example 16

### Preparation of DNT Oxalate

To a solution of 2.1 g of DNT-base (12% enantiomer R) dissolved in 12 ml of ethyl acetate was added a solution of 0.6 g of oxalic acid in 12 ml of ethyl acetate. The resulting mixture was stirred at room temperature for an hour, filtrated and washed with ethyl acetate. After drying, in a vacuum oven for overnight, 2 g (77% yield) of DNT-oxalate were obtained containing 12% of enantiomer R.

### Example 17

A 100 ml three necked flask, equipped with mechanical stirrer, thermometer, dean stark, and condenser, was charged with 5 g of DNT and 25 ml of toluene. The clear solution was heated, and an azeotropic distillation was performed for about 30 to about 60 minutes. After cooling to room temperature, 4.6 ml of ethyl chloroformate were added during over a period of 1 to 2 hours, and the reaction mixture was stirred at room temperature over night.

Diluted NH₄OH was added to the reaction mixture, which was stirred for an additional 30 minutes. After phase separation, the organic phase was washed with water (3×20 ml), dried over Na₂SO₄, filtered, and concentrated to dryness to give 5.2 g of a brownish oil. (88% chemical yield).

### Example 18

A 100 ml three necked flask equipped, with mechanical stirrer, thermometer, and condenser, was charged with 2.5 g duloxetine ethyl carbamate and 20 ml toluene. The mixture was stirred, and 4.8 g of KOH were added in portions, followed by reflux for about 3 hours.

After cooling, 30 ml of water, followed by 20 ml of toluene, were added, and the resulting organic phase was washed with water (3×20 ml), dried over Na₂SO₄, filtered and concentrated to dryness to give 1.70 g of an oily product. (85.31% yield).

### Example 19

To a solution of 1 g of duloxetine in 10 ml MEK was slowly added 0.32 ml of a 37 percent hydrochloric acid solution. The mixture was stirred until a solid formed. The resulting solid was filtered, and dried in a vacuum oven to give 0.50 g of (S)-(+)-duloxetine hydrochloride. (94.64% yield).

### Example 20

### Preparation of AT-ONE

A mixture of 50 g of 2-acetylthiophene (containing 0.56% 3-acetylthiophene), 42 g of dimethylamine hydrochloride, 18 g of paraformaldhyde, and 2 g of HCl [32%] in 125 ml IPA were heated to reflux for 4 hours. The mixture was cooled to 0° C., and the resulting solid was collected by filtration, washed with ethanol (125 ml×2), and used in the next step without further action.

### Example 21

### Preparation of rac-AT-OL

A solution of 90 g of AT-ONE from the previous example in 290 ml of methanol and 145 ml of water was cooled to 0° C. and 14 ml of NaOH [47%] were gradually added till pH 10. To the resulting solution was added portion added 12.1 g of sodium borohydride, and the mixture was allowed to warm to room temperature overnight. The methanol was evaporated under reduced pressure, and 250 ml were added, followed by the slow addition of concentrated HCl till pH 1.5, and stirred for an additional 20 minutes.

### Example 22

### Preparation of AT-OL-Mandelate

After basification with NaOH, the phases were separated, the water phase was washed with MTBE, and the combined organic phases were washed with brine. To the MTBE solution was added a solution of 16.4 g of (S)-mandelic acid in 40 ml ethanol, the resulting mixture was stirred at reflux for 1.25 hours, and then cooled to room temperature. The resulting solid was filtered, washed with MTBE, and dried in a vacuum oven to give 25 g of (S)-AT-OL mandelate.

### Example 23

### Preparation of AT-OL

To 20 g of AT-OL-mandelate in a mixture of 60 ml water and 90 ml MTBE were added NaOH [47%] till pH 9, and stirred at room temperature. After 30 minutes, the phases were separated, the organic phases were washed with water, and the residue evaporated to dryness.

### Example 24

### Preparation of DNT

To a solution of 7 g. of AT-OL in 42 ml of DMSO at room temperature were added 5 g of KOH, and stirred for an additional time. After 1 hour, 5 ml of 1-fluoronaphthalene were added, the solution was heated to 60° C., and stirred overnight.

To the reaction mixture was added water, followed by 80 ml HCl [5%], and extracted with 40 ml ethyl acetate (twice). After phase separation, the organic phase was washed with brine, and concentrated to dryness to give 10.5 g of brownish oil containing 0.12% of DNT-ISO3: 0.12%.

### Example 25

### Preparation of DNT-Maleate Free of DNT-ISO3

3.8 g of maleic acid were added to a solution of 10 g of DNT-base dissolved in 100 ml of ethyl acetate heated to reflux and cooled to room temperature. The resulting solid was filtered and washed with ethyl acetate. After drying in a vacuum oven at 50° C. for 16 hours, 5.5 g of DNT-maleate were obtained free of DNT-ISO3.

### Example 26

### Preparation of DNT Base Free of DNT-ISO3

A 2 liter reactor equipped with a mechanical stirrer is charged with a mixture of 107 g DNT-Maleate, 600 ml of water, 96 ml of a solution of ammonium hydroxide [22%], and 1 liter toluene. The mixture is stirred at 25° C. for 20-30 minutes, and the organic phase separated and washed with water (3×300 ml). The toluene solution containing the DNT-base free of DNT-ISO3 is evaporated to dryness.

### Example 27

### Preparation of (S)-Duloxetine Ethyl Carbamate

A 1 liter reactor, equipped with a mechanical stirrer, thermometer, dean stark, and condenser, is charged with (S)-DNT-base obtained in Example 6 dissolved in 1020 ml of toluene and 13 g of K₂CO₃. The mixture is heated, and an azeotropic distillation of 284 ml of the mixture is performed. After cooling to 50° C., 47.46 ml of ethyl chloroformate are added over a period of a half hour, and the reaction mixture is stirred at the same temperature for an additional 2 hours. After cooling to room temperature, the reaction mixture is washed with 230 ml of water, 130 ml of a 5 percent HCl solution, 130 ml of water, 130 ml of a 5 percent NaHCO₃ solution, and 130 ml of water. The resulting toluene solution of (S)-duloxetine ethyl carbamate is used in Example 9 without evaporation.

### Example 28

### Preparation of (S)-Duloxetine Base Free of DLX-ISO3

A 1 liter reactor, equipped with a mechanical stirrer, thermometer, and condenser, is charged with the solution of (S)-duloxetine ethyl carbamate in toluene prepared in Example 7. The mixture is heated, and an azeotropic distillation of 268 ml is performed. After cooling to 60° C., 82.18 g of an 85 percent KOH solution are added, and the mixture is heated to 94° C. for about 4 hours. After cooling to 60° C., 270 ml of water are added, and the resulting organic phase is washed three times with 270 ml of water, and treated with 4.6 g of charcoal (SX1) for 15 minutes, filtrated through a hyperflow bed, and washed with 60 ml of toluene. The solution is distillated at 30° to 40° C. under a vacuum of 20 to 30 mmHg until a volume of about 1 to 2 volumes of toluene is obtained.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

1-108. (canceled)

109. A process for preparing (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC comprising:
(a) combining (S)—N,N-dimethyl-3-(1-naphthalenyloxy)-3-(2-thienyl)propanamine and maleic acid to form a reaction mixture;
(b) precipitating (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC from the reaction mixture; and
(c) recovering the (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC from the reaction mixture.

110. The process of claim 109, wherein the (S)—N,N-dimethyl-3-(1-naphthalenyloxy)-3-(2-thienyl)propanamine maleate contains less than about 0.5% N,N-dimethyl-3-(1-naphthalenyloxy)-3-(3-thienyl)propanamine by HPLC.

111. The process of claim 109, wherein the (S)—N,N-dimethyl-3-(1-naphthalenyloxy)-3-(2-thienyl)propanamine maleate contains less than about 0.14% N,N-dimethyl-3-(1-naphthalenyloxy)-3-(3-thienyl)propanamine by HPLC.

112. The process of claim 109, wherein the (S)—N,N-dimethyl-3-(1-naphthalenyloxy)-3-(2-thienyl)propanamine maleate contains less than about 0.07% N,N-dimethyl-3-(1-naphthalenyloxy)-3-(3-thienyl)propanamine by HPLC.

113. The process of claim 109, wherein the (S)—N,N-dimethyl-3-(1-naphthalenyloxy)-3-(2-thienyl)propanamine maleate contains less than about 0.04% N,N-dimethyl-3-(1-naphthalenyloxy)-3-(3-thienyl)propanamine by HPLC.

114. The process of claim 109, wherein the (S)—N,N-dimethyl-3-(1-naphthalenyloxy)-3-(2-thienyl)propanamine maleate contains essentially 0.0% N,N-dimethyl-3-(1-naphthalenyloxy)-3-(3-thienyl)propanamine by HPLC.

115. A process for preparing (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC comprising:
(a) preparing (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC by the process of claim 109; and
(b) converting the (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC into (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC; and, optionally,
(c) converting the (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC into a salt of (S)—N,N-dimethyl-3-(1-naphthalenyloxy)-3-(2-thienyl)propanamine containing less than about 1% N,N-dimethyl-3-(1-naphthalenyloxy)-3-(3-thienyl)propanamine by HPLC.

116. The process of claim 115, wherein the (S)—N,N-dimethyl-3-(1-naphthalenyloxy)-3-(2-thienyl)propanamine or salt thereof contains less than about 0.5% N,N-dimethyl-3-(1-naphthalenyloxy)-3-(3-thienyl)propanamine by HPLC.

117. The process of claim 115, wherein the (S)—N,N-dimethyl-3-(1-naphthalenyloxy)-3-(2-thienyl)propanamine maleate contains less than about 0.14% N,N-dimethyl-3-(1-naphthalenyloxy)-3-(3-thienyl)propanamine by HPLC.

118. The process of claim 115, wherein the (S)—N,N-dimethyl-3-(1-naphthalenyloxy)-3-(2-thienyl)propanamine maleate contains less than about 0.07% N,N-dimethyl-3-(1-naphthalenyloxy)-3-(3-thienyl)propanamine by HPLC.

119. The process of claim 115, wherein the (S)—N,N-dimethyl-3-(1-naphthalenyloxy)-3-(2-thienyl)propanamine or salt thereof contains less than about 0.04% N,N-dimethyl-3-(1-naphthalenyloxy)-3-(3-thienyl)propanamine by HPLC.

120. The process of claim 115, wherein the (S)—N,N-dimethyl-3-(1-naphthalenyloxy)-3-(2-thienyl)propanamine or salt thereof contains essentially 0.0% N,N-dimethyl-3-(1-naphthalenyloxy)-3-(3-thienyl)propanamine by HPLC.

121. (S)—N,N-dimethyl-3-(1-naphthalenyloxy)-3-(2-thienyl)propanamine maleate containing less than about 1% N,N-dimethyl-3-(1-naphthalenyloxy)-3-(3-thienyl)propanamine by HPLC prepared by the process of claim 109.

122. (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC prepared by the process of claim 115.

123. (S)—N,N-dimethyl-3-(1-naphthalenyloxy)-3-(2-thienyl)propanamine or a salt thereof containing less than about 1% N,N-dimethyl-3-(1-naphthalenyloxy)-3-(3-thienyl)propanamine by HPLC.

124. The (S)—N,N-dimethyl-3-(1-naphthalenyloxy)-3-(2-thienyl)propanamine or salt thereof of claim 123 containing less than about 0.5% N,N-dimethyl-3-(1-naphthalenyloxy)-3-(3-thienyl)propanamine by HPLC.

125. The (S)—N,N-dimethyl-3-(1-naphthalenyloxy)-3-(2-thienyl)propanamine or salt thereof of claim 123 containing less than about 0.14% N,N-dimethyl-3-(1-naphthalenyloxy)-3-(3-thienyl)propanamine by HPLC.

126. The (S)—N,N-dimethyl-3-(1-naphthalenyloxy)-3-(2-thienyl)propanamine or salt thereof of claim 123 containing less than about 0.07% N,N-dimethyl-3-(1-naphthalenyloxy)-3-(3-thienyl)propanamine by HPLC.

127. (S)—N,N-dimethyl-3-(1-naphthalenyloxy)-3-(2-thienyl)propanamine or salt thereof of claim 123 containing less than about 0.04% N,N-dimethyl-3-(1-naphthalenyloxy)-3-(3-thienyl)propanamine by HPLC.

128. (S)—N,N-dimethyl-3-(1-naphthalenyloxy)-3-(2-thienyl)propanamine or salt thereof of claim 123 containing essentially 0.0% N,N-dimethyl-3-(1-naphthalenyloxy)-3-(3-thienyl)propanamine by HPLC.
